(19) United States
(12) Patent Application Publication
(10) Pub. No.: US 2009/0238867 A1
(43) Pub. Date: Sep. 24, 2009

(54) Nanoparticulate Anidulafungin Compositions and Methods for Making the Same

(76) Inventors: Scott Jenkins, Downingtown, PA (US); Gary Liversidge, West Chester, PA (US); Deborah Neville, Lower Gwynedd, PA (US)

Correspondence Address: Fox Rothschild, LLP;Elan Pharma International Limited, 2000 Market Street, Philadelphia, PA, 19103, US

(21) Appl. No.: 12/331,052

(22) Filed: Dec. 9, 2008

Related U.S. Application Data
(60) Provisional application No. 61/013,423, filed on Dec. 13, 2007.

(51) Int. Cl. A61K 9/66; A61K 9/16; A61K 9/52; A61K 9/26; A61K 9/20; A61P 31/10; A61K 38/12

(52) U.S. Cl. 424/455; 424/490; 424/497; 424/494; 424/493; 424/458; 424/469; 424/464; 514/9

(57) ABSTRACT

Nanoparticulate compositions comprising anidulafungin are described, as well as methods of making such compositions. Also described are methods for treatment of fungal infections.

### FIELD OF THE INVENTION

This application claims the priority benefit of the U.S. Provisional Application No. 61/013,423, filed on Dec. 13, 2007.

The present invention relates generally to nanoparticulate compositions of anidulafungin, and in particular, a nanoparticulate composition useful in the treatment of fungal infections.

### BACKGROUND OF THE INVENTION

The following discussion of the background of the invention is merely provided to aid the reader in understanding the invention and is not admitted to describe or constitute prior art to the invention.

### Background Regarding Anidulafungin

Anidulafungin is a semi-synthetic lipopeptide synthesized from a fermentation product of Aspergillus nidulans. Anidulafungin is an echinocandin, a class of antifungal drugs that inhibits the synthesis of 1,3-β-D-glucan, an essential component of fungal cell walls. Anidulafungin is 1-[(4R,5R)-4,5-Dihydroxy-N²-[[4″-(pentyloxy)[1,1′:4′,1″-terphenyl]-4-yl]carbonyl]-L-ornithine]echinocandin B. Anidulafungin is a white to off-white powder that is practically insoluble in water and slightly soluble in ethanol. The empirical formula of anidulafungin is C₅₈H₇₃N₇O₁₇ and the formula weight is 1140.3.

The structural formula is:

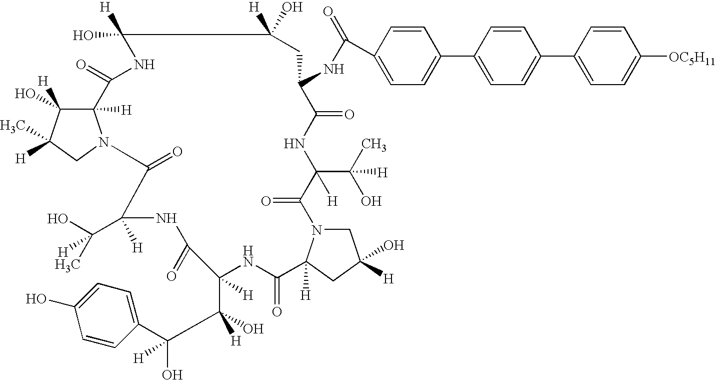

Anidulafungin is commercially available in the U.S. under the trade name ERAXIS® and is indicated for Candidemia and other forms of Candida infections such as intra-abdominal abscess, peritonitis, and esophageal candidiasis. It is distributed by Roerig, a division of Pfizer Inc. The anidulafungin compound, pharmaceutical formulations of anidulafungin and processes for making the same, and methods for inhibiting fungal or parasitic growth are described in U.S. Pat. Nos. 5,965,525; 6,384,013; 6,743,777; 6,960,564; and 7,198,796, each of which is hereby incorporated by reference.

Anidulafungin is a semi-synthetic echinocandin with antifungal activity. Anidulafungin inhibits glucan synthase, an enzyme present in fungal, but not mammalian cells. This results in inhibition of the formation of 1,3-β-D-glucan, an essential component of the fungal cell wall. Anidulafungin is active in vitro against Candida albicans, C. glabrata, C. parapsilosis, and C. tropicalis. Parenterally administered anidulafungin is effective against Candida albicans in immunocompetent and immunosuppressed mice and rabbits with disseminated infection as measured by prolonged survival and reduction in mycological burden. Anidulafungin also reduces the mycological burden of fluconazole-resistant C. albicans in an oropharyngeal/esophageal infection model in immunosuppressed rabbits.

The pharmacokinetics of anidulafungin following IV administration have been characterized in healthy subjects, special populations and patients. Systemic exposures of anidulafungin are dose proportional and have low intersubject variability (coefficient of variation <25%). The steady state is achieved on the first day after a loading dose (twice the daily maintenance dose) and the estimated plasma accumulation factor at steady state is approximately 2. The clearance of anidulafungin is about 1 L/h and anidulafungin has a terminal elimination half-life of 40-50 hours. The pharmacokinetics of anidulafungin following IV administration are characterized by a short distribution half-life (0.5-1 hour) and a volume of distribution of 30-50 L that is similar to total body fluid volume. Anidulafungin is extensively bound (>99%) to human plasma proteins.

Hepatic metabolism of anidulafungin has not been observed. Anidulafungin is not a clinically relevant substrate, inducer, or inhibitor of cytochrome P450 (CYP450) isoenzymes. It is unlikely that anidulafungin will have clinically relevant effects on the metabolism of drugs metabolized by CYP450 isoenzymes. Anidulafungin undergoes slow chemical degradation at physiologic temperature and pH to a ring-opened peptide that lacks antifungal activity. The in vitro degradation half-life of anidulafungin under physiologic conditions is about 24 hours. In vivo, the ring-opened product is subsequently converted to peptidic degradants and eliminated.

As currently formulated (ERAXIS®), anidulafungin must be administered daily as a slow IV infusion (<1.1 mg/min) for at least 14 days after the last positive culture for candidemia and other Candida infections (intra-abdominal abscess and peritonitis) and for at least 7 days following the resolution of symptoms for esophageal candidiasis. Thus, the administration of anidulafungin is cumbersome and is very inconvenient for the patient. Accordingly, an intramuscular depot formulation of anidulafungin that could be administered once a week, once every two weeks, once every three weeks, or once every four weeks would be desirable.

### Background Regarding Nanoparticulate Compositions

Nanoparticulate compositions, first described in U.S. Pat. No. 5,145,684 (“the '684 patent”), hereby incorporated by reference, are particles consisting of a poorly soluble therapeutic or diagnostic agent having adsorbed onto the surface thereof a non-crosslinked surface stabilizer. The '684 patent does not describe nanoparticulate compositions of anidulafungin, its enantiomers, or polymorphs.

Methods of making nanoparticulate compositions are described in, for example, U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of the above patents are incorporated by reference, as are all the earlier aforementioned patents.

Nanoparticulate compositions are also described, for example, in U.S. Pat. No. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydroxypropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” U.S. Pat. No. 6,270,806 for “Use of PEG-Derivatized Lipids as Surface Stabilizers for Nanoparticulate Compositions;” U.S. Pat. No. 6,316,029 for “Rapidly Disintegrating Solid Oral Dosage Form,” U.S. Pat. No. 6,375,986 for “Solid Dose Nanoparticulate Compositions Comprising a Synergistic Combination of a Polymeric Surface Stabilizer and Dioctyl Sodium Sulfosuccinate;” U.S. Pat. No. 6,428,814 for “Bioadhesive Nanoparticulate Compositions Having Cationic Surface Stabilizers;” U.S. Pat. No. 6,431,478 for “Small Scale Mill;” and U.S. Pat. No. 6,432,381 for “Methods for Targeting Drug Delivery to the Upper and/or Lower Gastrointestinal Tract,” all of which are specifically incorporated by reference. In addition, U.S. Patent Application No. 20020012675 A1, published on Jan. 31, 2002, for “Controlled Release Nanoparticulate Compositions,” describes nanoparticulate compositions, and is specifically incorporated by reference, as are all the earlier aforementioned patents.

Amorphous small particle compositions are described, for example, in U.S. Pat. No. 4,783,484 for “Particulate Composition and Use Thereof as Antimicrobial Agent;” U.S. Pat. No. 4,826,689 for “Method for Making Uniformly Sized Particles from Water-Insoluble Organic Compounds;” U.S. Pat. No. 4,997,454 for “Method for Making Uniformly-Sized Particles From Insoluble Compounds;” U.S. Pat. No. 5,741,522 for “Ultrasmall, Non-aggregated Porous Particles of Uniform Size for Entrapping Gas Bubbles Within and Methods;” and U.S. Pat. No. 5,776,496, for “Ultrasmall Porous Particles for Enhancing Ultrasound Back Scatter,” which are also hereby incorporated by reference herein.

The present invention relates, in certain aspects, to nanoparticulate compositions comprising anidulafungin or an analogue thereof, which may be useful in the treatment of fungal infections as well as methods for making and using the same.

### SUMMARY OF THE INVENTION

The present invention relates to nanoparticulate compositions comprising anidulafungin or a salt, derivative, conjugate, hydrate, polymorph or analogue thereof, as well as methods of making and using the same.

In one aspect of the invention, the composition comprises particles comprising anidulafungin, wherein the particles have an average size of less than about 2000 nm. The composition may also comprise at least one surface stabilizer adsorbed onto or associated with the surface of the particles.

Further aspects of present invention relate to dosage forms made from the compositions of the present invention. In one embodiment, the nanoparticulate composition is formulated for oral delivery. In another embodiment, the nanoparticulate composition is an injectable formulation. A preferred dosage form of the invention is a subcutaneous or intramuscular depot for long term release. Another preferred dosage form of the invention is a formulation suitable for intravenous administration that can be infused more rapidly than the current commercial formulation. In another embodiment, the nanoparticulate composition is formulated for ocular administration. In another embodiment, the nanoparticulate is formulated for pulmonary administration.

Further aspects of the invention are directed to methods of making compositions according to the invention. According to one aspect of the invention, a method for making a nanoparticulate anidulafungin composition comprises the step of contacting at least one active agent selected from the group consisting of anidulafungin, salts of anidulafungin, derivatives of anidulafungin, conjugates of anidulafungin, hydrates of anidulafungin, polymorphs of anidulafungin, and analogues of anidulafungin, with at least one surface stabilizer for a period of time and under conditions sufficient to provide a nanoparticulate composition having an effective average particle size of less than about 2000 nm.

Additional aspects of the present invention are directed to methods of treating certain conditions comprising administering an effective amount of a nanoparticulate composition comprising anidulafungin or a salt, derivative, conjugate, hydrate, polymorph or analogue thereof to a subject in need thereof.

Both the foregoing general description and the following detailed description are exemplary and explanatory, and are intended to provide further explanation of the invention as claimed. Other objects, advantages, and novel features will be readily apparent to those skilled in the art from the following detailed description of the invention.

### DETAILED DESCRIPTION OF THE INVENTION

### Definitions

As employed above and throughout the disclosure, the following terms, unless otherwise indicated, shall be understood to have the following meanings.

As used herein, “about” will be understood by persons of ordinary skill in the art and will vary to some extent according to the context in which it is used. If there are uses of the term which are not clear to persons of ordinary skill in the art given the context in which it is used, “about” will mean up to plus or minus 10% of the particular term.

As used herein, “particulate” refers to a state of matter which is characterized by the presence of discrete particles, pellets, beads or granules irrespective of their size, shape or morphology.

As used herein, “nanoparticulate” refers to a composition in which the effective average particle size of the particles therein is less than about 2000 nm (2 microns).

As used herein, the terms “conventional” or “non-nanoparticulate” active agent shall mean an active agent, such as anidulafungin or analogue thereof, which is solubilized or which has an effective average particle size of greater than about 2000 nm.

As used herein, “effective average particle size” describes a population of particles in a composition in which 50% of the particles are less than a specified size. Accordingly, “effective average particle size of less than about 2000 nm” means that at least 50% of the particles therein are less than about 2000 nm.

As used herein, “D50” refers to a particle size below which 50% of the particles in a composition are less than that particle size. Similarly, “D90” refers to the particle size below which 90% of the particles in a composition are less than that particle size.

As used herein with reference to stable particles, “stable” refers to, but is not limited to, one or more of the following parameters: (1) the particles do not appreciably flocculate or agglomerate due to interparticle attractive forces or otherwise significantly increase in particle size over time; (2) the physical structure of the particles is not altered over time, such as by conversion from an amorphous phase to a crystalline phase; (3) the particles are chemically stable; and/or (4) where the active ingredient has not been subject to a heating step at or above the melting point of the active agent in the preparation of the particles of the present invention.

As used herein, “poorly water soluble drug” refers to a drug that has a solubility in water of less than about 30 mg/ml, less than about 20 mg/ml, less than about 10 mg/ml, or less than about 1 mg/ml.

As used herein, “therapeutically effective amount” means the dosage that provides the specific pharmacological response for which the active agent is administered in a significant number of subjects in need of the relevant treatment. It is emphasized that a therapeutically effective amount of the active agent that is administered to a particular subject in a particular instance will not always be effective in treating the conditions described herein, even though such dosage is deemed to be a therapeutically effective amount by those of skill in the art.

As used herein, the term “anidulafungin” or “active agent” includes anidulafungin, as well as salts, derivatives, conjugates, hydrates, polymorphs, and analogues thereof. Anidulafungin or an analogue thereof may be present either in the form of one substantially optically pure enantiomer or as a mixture, racemic or otherwise, of enantiomers.

The terms “sterilize” or “sterilized” as used in the present application generally means to inactivate biological contaminants present in the product. In typical pharmaceutical applications, exposure to at least a 25 kGray dose of irradiation sterilizes the pharmaceutical product or sterile filtered through a 0.2 micron sieve.

### Nanoparticulate Compositions

In one aspect of the invention, the composition comprises particles comprising anidulafungin wherein the particles have an effective average particle size of less than about 2000 nm; and at least one surface stabilizer adsorbed on a surface of the particles.

The nanoparticulate particles described herein may exist in a crystalline phase, an amorphous phase, a semi-crystalline phase, a semi amorphous phase, or a mixture thereof.

Anidulafungin is selected from the group consisting of anidulafungin, salts of anidulafungin, derivatives of anidulafungin, conjugates of anidulafungin, hydrates of anidulafungin, polymorph of anidulafungin, analogues of anidulafungin, and mixtures thereof.

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

Compositions of the invention comprise anidulafungin or analogue thereof particles having an effective average particle size of less than about 2 microns. In other embodiments of the invention, the anidulafungin or analogue thereof particles have an effective average particle size of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

In another embodiment of the invention, the compositions of the invention are in an injectable dosage form and the anidulafungin or analogue thereof particles have an effective average particle size of less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or less than about 50 nm. Injectable compositions can comprise anidulafungin or an analogue thereof having an effective average particle size of greater than about 1 micron, up to about 2 microns. If the “effective average particle size” is less than about 600 nm, then at least about 50% of the anidulafungin or analogue thereof particles have a size of less than about 600 nm, when measured by the above-noted techniques. The same is true for the other particle sizes referenced above.

In certain embodiments, at least about 60%, at least about 70%, at least about at least about 80%, at least about 90%, at least about 95%, or at least about 99% of the anidulafungin or analogue thereof particles have a particle size less than the effective average, i.e., less than about 1000 nm, less than about 900 nm, less than about 800 nm, etc. In other embodiments, at least about 60%, at least about 70%, at least about 80%, at least about 90%, at least about 95%, or at least about 99% of the redispersed active agent particles have a particle size of less than the effective average, i.e., less than about 2000 nm, less than about 1900 nm, less than about 1800 nm, less than about 1700 nm, etc.

In certain aspects, the invention provides compositions comprising nanoparticulate anidulafungin or analogue thereof particles and at least one surface stabilizer. The surface stabilizers are preferably adsorbed onto or associated with the surface of the anidulafungin or analogue thereof particles. Surface stabilizers useful herein may physically adhere on or associate with the surface of the nanoparticulate active agent but may not chemically react with the active agent particles. In another embodiment, the compositions of the present invention may comprise two or more surface stabilizers.

Exemplary useful surface stabilizers include, but are not limited to, known organic and inorganic pharmaceutical excipients, as well as peptides and proteins. Such excipients include various polymers, low molecular weight oligomers, natural products, and surfactants. Useful surface stabilizers include nonionic surface stabilizers, ionic surface stabilizers, cationic surface stabilizers, anionic surface stabilizers, and zwitterionic surface stabilizers. Combinations of more than one surface stabilizer may be used in the invention.

Representative examples of surface stabilizers include, but are not limited to, hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Specialty Chemicals)); polyethylene glycols (e.g., Carbowaxes 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-lOG® or Surfactant 10-G® (Olin Chemicals, Stamford, Conn.); Crodestas SL-40® (Croda, Inc.); and SA90HCO, which is C18H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, and the like.

Additional examples of useful surface stabilizers include, but are not limited to, polymers, biopolymers, polysaccharides, cellulosics, alginates, phospholipids, and nonpolymeric compounds, such as zwitterionic stabilizers, poly-n-methylpyridinium chloride, anthryul pyridinium chloride, cationic phospholipids, chitosan, polylysine, polyvinylimidazole, polybrene, polymethylmethacrylate trimethylammonium bromide (PMMTMABr), hexyldecyltrimethylammonium bromide (HDMAB), and polyvinylpyrrolidone-2-dimethylaminoethyl methacrylate dimethyl sulfate.

Other useful stabilizers include, but are not limited to, cationic lipids, sulfonium, phosphonium, and quaternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl(C₁₂₋₁₈)dimethylbenzyl ammonium chloride, N-alkyl(C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990), all of which are incorporated herein by reference.

Other known pharmaceutical excipients which may be suitable as surface stabilizers are described in detail in the Handbook of Pharmaceutical Excipients, 4th Edition, 2003, published jointly by the American Pharmaceutical Association and The Pharmaceutical Society of Great Britain (Pharmaceutical Press), specifically incorporated by reference herein. Pharmaceutical excipients listed therein include: acacia, acesulfame potassium, albumin, alcohol, alginic acid, aliphatic polyesters, alpha tocopherol, ascorbic acid, ascorbyl palmitate, aspartame, bentonite, benzalkonium chloride, benzethonium chloride, benzoic acid, benzyl alcohol, benzyl benzoate, bronopol, butylated hydroxyanisole, butylated hdroxytoluene, butylparaben, calcium carbonate, calcium phosphate dibasic anhydrous, calcium phosphate dibasic dehydrate, calcium phosphate tribasic, calcium stearate, calcium sulfate, canola oil, carbomer, carbon dioxide, carboxymethylcellulose calcium, carboxymethylcellulose sodium, carrageenan, hydrogenated castor oil, cellulose acetate, cellulose acetate phthalate, microcrystalline cellulose, powdered cellulose, silicified microcrystalline cellulose, cetostearyl alcohol, cetrimide, cetyl alcohol, chlorhexidine, chlorobutanol, chlorocresol, chlorodifluoroethane (HCFC), chlorofluorocarbons (CFC), cholesterol, citric acid monohydrate, colloidal silicon dioxide, coloring agents, corn oil, cottonseed oil, cresol, croscarmellose sodium, crospovidone, cyclodextrins, dextrates, dextrin, dextrose, dibutyl sebacate, diethanolamine, diethyl phthalate, difluoroethane (HFC), dimethyl ether, docusate sodium, edetic acid, ethylcellulose, ethyl maltol, ethyl oleate, ethylparaben, ethyl vanillin, fructose, fumaric acid, gelatin, liquid glucose, glycerin, glyceryl monooleate, glyceryl monostearate, glyceryl palmitostearate, glycofurol, guar gum, heptafluoropropane (HFC), hydrocarbons (HC), hydrochloric acid, hydroxyethyl cellulose, hydroxypropyl cellulose, low-substituted hydroxypropyl cellulose, hydroxypropyl methylcellulose, hydroxypropyl methylcellulose phthalate, imidurea, isopropyl alcohol, isopropyl myristate, isopropyl palmitate, kaolin, lactic acid, lactitol, lactose, lanolin, lanolin alcohols, hydrous lanolin, lecithin, magnesium aluminum silicate, magnesium carbonate, magnesium oxide, magnesium stearate, magnesium trisilicate, malic acid, maltitol, maltitol solution, maltodextrin, maltol, maltose, mannitol, medium chain triglycerides, meglumine, menthol, methylcellulose, methylparaben, mineral oil, light mineral oil, mineral oil and lanolin alcohols, monoethanolamine, nitrogen, nitrous oxide, oleic acid, paraffin, peanut oil, petrolatum, petrolatum and lanolin alcohols, phenol, phenoxyethanol, phenylethyl alcohol, phenylmercuric acetate, phenylmercuric borate, phenylmercuric nitrate, polacrilin potassium, poloxamer, polydextrose, polyethylene glycol, polyethylene oxide, polymethacrylates, polyoxyethylene alkyl ethers, polyoxyethylene castor oil derivatives, polyoxyethylene sorbitan fatty acid esters, polyoxyethylene stearates, polyvinyl alcohol, potassium chloride, potassium citrate, potassium sorbate, povidone, propylene carbonate, propylene glycol, propylene glycol alginate, propyl gallate, propylparaben, saccharin, saccharin sodium, sesame oil, shellac, sodium alginate, sodium ascorbate, sodium benzoate, sodium bicarbonate, sodium chloride, sodium citrate dihydrate, sodium cyclamate, sodium lauryl sulfate, sodium metabisulfite, dibasic sodium phosphate, monobasic sodium phosphate, sodium propionate, sodium starch glycolate, sodium stearyl fumarate, sorbic acid, sorbitan esters (sorbitan fatty acid esters), sorbitol, soybean oil, starch, pregelatinized starch, sterilizable maize starch, stearic acid, stearyl alcohol, sucrose, compressible sugar, confectioner's sugar, sugar spheres, hard fat suppository bases, talc, tartaric acid, tetrafluoroethane (HFC), thimerosal, titanium dioxide, tragacanth, triacetin, triethanolamine, triethyl citrate, vanillin, type I hydrogenated vegetable oil, water, anionic emulsifying wax, Carnauba wax, cetyl esters wax, microcrystalline wax, nonionic emulsifying wax, white wax, yellow wax, xanthan gum, xylitol, zein, and zinc stearate.

In certain embodiments of the invention, the composition may comprise at least one peptide as a surface stabilizer adsorbed on to, or associated with, the surface of the active agent. The peptide surface stabilizer can be contacted with the active agent before, preferably during, or after size reduction of the active agent.

In certain other embodiments of the invention, the composition may comprise at least one protein as a surface stabilizer. As a non-limiting example, compositions according to certain embodiments of the invention may comprise an albumin, for example, human serum albumin.

The relative amounts of anidulafungin or analogue thereof and one or more surface stabilizers can vary widely. The optimal amount of the individual components depends, for example, upon physical and chemical attributes of the surface stabilizer(s) and anidulafungin or analogue thereof selected, such as the hydrophilic lipophilic balance (HLB), melting point, and the surface tension of water solutions of the stabilizer, etc.

Preferably, the concentration of the anidulafungin or analogue thereof can vary from about 99.5% to about 0.001%, from about 95% to about 0.1%, or from about 90% to about 0.5%, by weight, based on the total combined weight of the anidulafungin or analogue thereof and at least one surface stabilizer, not including other excipients. Higher concentrations of the active ingredient are generally preferred from a dose and cost efficiency standpoint.

Preferably, the concentration of surface stabilizer can vary from about 0.5% to about 99.999%, from about 5.0% to about 99.9%, or from about 10% to about 99.5%, by weight, based on the total combined dry weight of the anidulafungin or analogue thereof and at least one surface stabilizer, not including other excipients.

Certain embodiments of the invention may include nanoparticulate anidulafungin or analogue thereof compositions together with one or more non-toxic physiologically acceptable excipients, carriers, adjuvants, or vehicles, collectively referred to as carriers. The compositions may be formulated, for example, for parenteral injection (e.g., intravenous, intramuscular, intrathecal, or subcutaneous), oral administration in solid, liquid, or aerosol form, vaginal, nasal, pulmonary, rectal, ocular, local (powders, ointments or drops), buccal, intracisternal, intraperitoneal, or topical administration, and the like.

Non-limiting examples of excipients that may be included in the composition are bulking agents, crystal growth inhibitors, free radical scavenger agents, and redispersion agents. Preferably, the excipient may be present in an amount from about 1 to about 50, about 1 to about 40, about 1 to about 30, about 1 to about 20, about 1 to about 15, about 1 to about 10, or about 1 to about 5, as measured by % w/w of the composition.

In certain embodiments, the compositions of the present invention may comprise also one or more binding agents, filling agents, diluents, lubricating agents, emulsifying and suspending agents, sweeteners, flavoring agents, preservatives, buffers, wetting agents, disintegrants, effervescent agents, perfuming agents, and other excipients. Such excipients are known in the art. In addition, prevention of the growth of microorganisms may be ensured by the addition of various antibacterial and antifungal agents, such as, for example, parabens, chlorobutanol, phenol, sorbic acid, and the like. For use in injectable formulations, the composition may comprise also isotonic agents, such as sugars, sodium chloride, and the like and agents for use in delaying the absorption of the injectable pharmaceutical form, such as, for example, aluminum monostearate and gelatin.

Compositions suitable for parenteral injection may comprise, for example, physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents, or vehicles including water, ethanol, sodium chloride, Ringer's solution, lactated Ringer's solution, stabilizer solutions, tonicity enhancers (sucrose, dextrose, mannitol, etc.) polyols (propyleneglycol, polyethylene-glycol, glycerol, and the like), suitable mixtures thereof, vegetable oils (such as olive oil) and injectable organic esters such as ethyl oleate. A number of fluids which may be suitable are referenced in Remington's Pharmaceutical Sciences, 17th edition, published by Mack Publishing Co., page 1543.

Exemplary preservatives useful in certain embodiments of the invention include, without limitation, methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

In certain embodiments, the compositions of the invention may comprise, in addition to anidulafungin or an analogue thereof, one or more compounds useful in treating various types of fungal or parasitic infections. Representative examples include, but are not limited to, 3M 003, 3-methoxysampangine, 3-methylsampangine, 4-aminosampangine, 4-methoxysampangine, abafungin, adrenomedullin peptides, ajoene, albaconazole, aminocandin, amorolfine, Amphotericin B, AN 2690, arasertaconazole, asperfuran, atpenin B, BAL 8349, basifungin, bis-pyridinum salts, BMS 379224, brefeldin A, butenafine, C 31G, CAN 296, caspofungin, CAY 1, CC 0262, cepacidine A, chitinase, Ciclopirox, clotrimazole, crocacin, CYD 1274, CZEN 002, darlucin A, DB 368, docosanol, eberconazole, ECO 2301, efungumab, embeconazole, ETD 151, ETS 4103, EV 086, fenticonazole, fluconazole, flutrimazole, fosfluconazole, FR 901469, FX 0549, FX 0685, G 1, GL 047296, GL 48656, GL 663142, GL 886217, GM 191519, GM 193663, GM 237354, Griseofulvin, HB 666, hLF 1-11, HWY 289, icofungipen, IDEA 067, interferon gamma-1b, isavuconazonium chloride, ITF 2534, itraconazole, jasplakinolide, KB 205, Ketoconazole, KP 103, L 693989, L 705589, L 731373, L 733560, lanimostim, lanoconazole, liranaftate, luliconazole, LY 307853, MAb, 2H1, MAb, rhenium-188, Cryptococcus, neoformans, Martek 92211, MER WF3010, meridine, MGCD 290, micafungin, MM 86553, MNLP 1250, MQX 5855, MRLP 098, MS 8209, MUC7 20-mer, Natamycin, NC 1175, N-chlorotaurine, neticonazole, NK 372135A, NK 372135B, NK 372135C, NVC 320, Nystatin, NZ 3000, ofloxacin, omoconazole, oxiconazole, P 1639C, PAC 113, pafuramidine, pneumocandin Do, posaconazole, pramiconazole, R 102557, ravuconazole, RBx 6510, RBx 7635, RBx 9050, rhMBL, NatImmune, rimoprogin, RLP 068, Ro 425604, Ro 430688, RS 135853, SCH 42137, SCH 59884, SEP 98035, sertaconazole, SPA S753, SPA S843, spartanamicin B, sphingofungin B, SPK 843, SQ 109, SQ 609, SS 750, SSY 726, ST 1103, ST 41517, ST 61219, ST 61769, synerazol, T 2307, T 8581, TAK 456, TAK 457, tebipenem pivoxil, terbinafine, Terbinafine, thymalfasin, TKR 1785, trimetrexate, V 253, V 283B methyl ester, VAGIPREV, voriconazole.

### Characteristics of Nanoparticulate Compositions

According to certain aspects of the invention, nanoparticulate compositions of the invention are proposed to have an unexpectedly rapid dissolution profile. Rapid dissolution of anidulafungin or an analogue thereof is preferable, as faster dissolution generally leads to faster onset of action and greater bioavailability. To improve the dissolution profile and bioavailability of the anidulafungin or an analogue thereof, it would be useful to increase the drug's dissolution so that it could attain a level close to 100% dissolved.

According to certain embodiments of the invention, compositions of the invention preferably have a dissolution profile in which within about 5 minutes at least about 20% of the anidulafungin or an analogue thereof is dissolved. In other embodiments of the invention, at least about 30% or at least about 40% of the anidulafungin or an analogue thereof is dissolved within about 5 minutes. In yet other embodiments of the invention, preferably at least about 40%, at least about 50%, at least about 60%, at least about 70%, or at least about 80% of the anidulafungin or an analogue thereof is dissolved within about 10 minutes. Finally, in another embodiment of the invention, preferably at least about 70%, at least about 80%, at least about 90%, or at least about 100% of the anidulafungin or an analogue thereof is dissolved within about 20 minutes.

Dissolution is preferably measured in a medium which is discriminating. Such a dissolution medium will produce two very different dissolution curves for two products having very different dissolution profiles in gastric juices; i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

In one embodiment of the invention, the nanoparticulate particles of the composition redisperse so that the particles have an effective average particle size of less than about 2000 nm. This is significant because, if the particles did not redisperse so that they have an effective average particle size of less than about 2000 nm, the composition may lose benefits afforded by formulating the anidulafungin or an analogue thereof therein into a nanoparticulate form. This is because nanoparticulate compositions benefit from the small size of the particles comprising the anidulafungin or an analogue thereof. If the particles do not redisperse into small particle sizes upon administration, then “clumps” or agglomerated particles may be formed, owing to the extremely high surface free energy of the nanoparticulate system and the thermodynamic driving force to achieve an overall reduction in free energy. With the formation of such agglomerated particles, the bioavailability of the dosage form may fall well below that observed with the liquid dispersion form of the nanoparticulate composition.

In other embodiments of the invention, the redispersed particles of the invention (redispersed in water, a biorelevant media, or any other suitable liquid media) have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm in diameter, as measured by light-scattering methods, microscopy, or other appropriate methods. Such methods suitable for measuring effective average particle size are known to a person of ordinary skill in the art.

Redispersibility can be tested using any suitable means known in the art. See e.g., the example sections of U.S. Pat. No. 6,375,986 for “Solid Dose Nanoparticulate Compositions Comprising a Synergistic Combination of a Polymeric Surface Stabilizer and Dioctyl Sodium Sulfosuccinate,” incorporated herein by reference.

### Pharmaceutical Formulations

According to certain aspects of the invention, the compositions may be formulated for administration via any pharmaceutically acceptable route of administration, including, but not limited to, parenteral, oral, pulmonary, rectal, ocular, colonic, intracisternal, intravaginal, intraperitoneal, local, buccal, nasal, and topical administration.

In certain aspects of the invention, the composition may be formulated into any pharmaceutically acceptable dosage form, including, but not limited to, liquid dispersions, solid dispersions, liquid-filled capsule, gels, aerosols, ointments, depots, creams, lyophilized formulations, tablets, capsules, multi-particulate filled capsule, tablet composed of multi-particulates, compressed tablet, and a capsule filled with enteric-coated beads of the active ingredient.

In another aspect of the invention, the composition may be formulated into dosage forms including, but not limited to, controlled release formulations, fast melt formulations, delayed release formulations, extended release formulations, pulsatile release formulations, and mixed immediate release and controlled release formulations, or any combination thereof.

Dosage forms that are preferably sterile include, but are not limited to, aerosols for nasal or pulmonary delivery, injectable, and ocular dosage forms.

In one embodiment of the invention, provided are injectable nanoparticulate anidulafungin or analogue thereof formulations that can comprise high concentrations in low injection volumes, with rapid dissolution upon administration, which can be infused more rapidly than current commercial formulations.

Exemplary preservatives useful with injectable formulations of the invention include, without limitation, methylparaben (about 0.18% based on % w/w), propylparaben (about 0.02% based on % w/w), phenol (about 0.5% based on % w/w), and benzyl alcohol (up to 2% v/v). An exemplary pH adjusting agent is sodium hydroxide, and an exemplary liquid carrier is sterile water for injection. Other useful preservatives, pH adjusting agents, and liquid carriers are well-known in the art.

Compositions suitable for parenteral injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

In certain embodiments of the invention, the nanoparticulate anidulafungin or analogue thereof composition, including an injectable composition, is free of polysorbate, ethanol, or a combination thereof. In addition, when formulated into an injectable formulation, the compositions of the invention may provide a high concentration in a small volume to be injected. Injectable anidulafungin or analogue thereof compositions of the invention can be administered, for example, in a bolus injection or with a slow infusion over a suitable period of time.

In certain embodiments of the invention, the nanoparticulate anidulafungin compositions are formulated as a subcutaneous or intramuscular depot. The depot is preferably formulated to release anidulafungin over a period from about one week to about four weeks. In other embodiments of the invention, the injectable depot nanoparticulate anidulafungin composition provides therapeutic levels of drug for up to about one week, up to about two weeks, up to about three weeks, or up about four weeks.

In another embodiment of the present invention, the nanoparticulate anidulafungin compositions are ocular formulations such as eyedrops (e.g. aqueous liquid suspensions). Suitable eyedrop formulations are those which are approximately isotonic and maintain sufficient contact with the eye surface to systemically deliver the active agent to the patient. Such formulations advantageously have a pH approximating neutrality and are non-irritating to the eye, e.g. they do not induce tearing and consequential flow of active agent out of the eye. Pharmaceutically acceptable carriers are, for example, water, mixtures of water and water-miscible solvents such as lower alkanols or arylalkanols, vegetable oils, polyalkylene glycols, petroleum based jelly, ethyl cellulose, hydroxy ethyl cellulose, ethyl oleate, carboxymethylcellulose, polyvinylpyrrolidone, isopropyl myristate and other conventionally-employed non-toxic, pharmaceutically acceptable organic and inorganic carriers. The pharmaceutical preparation may also contain non-toxic auxiliary substances such as emulsifying, preserving, wetting agents, bodying agents and the like, as for example, polyethylene glycols 200, 300, 400 and 600, carbowaxes 1000, 1500, 4000, 6000 and 10000, antibacterial compounds, phenylmercuric salts known to have cold sterilizing properties and which are non-injurious in use, thimerosal, methyl and propyl paraben, benzyl alcohol, phenyl ethanol, buffering ingredients such as sodium chloride, sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetraacetic acid, and the like. Additionally, suitable ophthalmic vehicles can be used as carrier media for the present purpose including conventional phosphate buffer vehicle systems, isotonic boric acid vehicles, isotonic sodium chloride vehicles, isotonic sodium borate vehicles and the like.

In the procedure for making eyedrops, formulations are rendered sterile by appropriate means, such as starting the preparation procedure with sterile components and proceeding under sterile conditions, irradiating or autoclaving the finished formulation, and the like. Suitable anti microbial agents are also useful for maintaining sterility of the eyedrop. Terminal sterilization may also be achieved by sterile filtration through a 0.2 micron sieve.

The ocular preparation may also be an ointment which is compounded, for example, by mixing finely milled powdered ingredients with a small amount of white petrolatum and levigating or otherwise mixing until a uniform distribution is achieved. The balance of white petrolatum is added by geometric addition until the desired dosage form is made.

In another embodiment of the present invention, the nanoparticulate anidulafungin compositions can be formulated into an inhalation formulation in the form of a sterile dispersion or suspension, wherein a composition according to the invention is a liquid for delivery of aqueous droplets comprising a anidulafungin nanoparticles via a nebulizer to the pulmonary system (e.g. bronchial system and lungs). It is also envisioned that for inhalation, the sterile dispersion or suspension of a composition according to the invention may be utilized in combination with other liquids and excipients and optionally a propellant for delivery via a metered dose inhaler (MDI) to the pulmonary system. It is further envisioned that for inhalation, the sterile dispersion or suspension of a composition according to the invention may be utilized with other liquids or excipients and converted to a dry powder alone for delivery via a dry powder inhaler (DPI) to the pulmonary system (see e.g., US 20020102294 A1 to Bosch et al., for “Aerosols Comprising Nanoparticle Drugs”). Sterile nasal formulations can be in the form of a solution of a composition according to the invention in an appropriate liquid phase with additional excipients and stabilizers as required.

### Methods of Making Nanoparticulate Anidulafungin Formulations

According to certain aspects of the invention, nanoparticulate active agent compositions can be made using methods known in the art such as, for example, milling, homogenization, and precipitation techniques. Exemplary methods of making nanoparticulate active agent compositions are generally described in U.S. Pat. No. 5,145,684 (“the '684 patent”), the contents of which are incorporated by reference herein. The '684 patent describes nanoparticles of poorly soluble therapeutic or diagnostic agents having adsorbed onto or associated with the surface thereof a non-crosslinked surface stabilizer.

Methods of making nanoparticulate active agent compositions are also described in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” U.S. Pat. No. 5,665,331, for “Co-Microprecipitation of Nanoparticulate Pharmaceutical Agents with Crystal Growth Modifiers;” U.S. Pat. No. 5,662,883, for “Co-Microprecipitation of Nanoparticulate Pharmaceutical Agents with Crystal Growth Modifiers;” U.S. Pat. No. 5,560,932, for “Microprecipitation of Nanoparticulate Pharmaceutical Agents;” U.S. Pat. No. 5,543,133, for “Process of Preparing X-Ray Contrast Compositions Containing Nanoparticles;” U.S. Pat. No. 5,534,270, for “Method of Preparing Stable Drug Nanoparticles;” U.S. Pat. No. 5,510,118, for “Process of Preparing Therapeutic Compositions Containing Nanoparticles;” and U.S. Pat. No. 5,470,583, for “Method of Preparing Nanoparticle Compositions Containing Charged Phospholipids to Reduce Aggregation,” all of which are incorporated herein by reference.

In one embodiment of the invention, particles comprising anidulafungin or an analogue thereof may be dispersed in a liquid dispersion medium in which the anidulafungin, or analogue thereof, is poorly soluble. Mechanical means are then used in the presence of grinding media to reduce the particle size to the desired effective average particle size. The dispersion medium can be, for example, water, safflower oil, ethanol, t-butanol, glycerin, polyethylene glycol (PEG), hexane, or glycol. A preferred dispersion medium is water. The particles can be reduced in size in the presence of at least one surface stabilizer. The particles comprising anidulafungin or an analogue thereof can be contacted with one or more surface stabilizers after attrition. Other compounds, such as a diluent, can be added to the composition during the size reduction process. Dispersions can be manufactured continuously or in a batch mode. One skilled in the art would understand that it may be the case that, following milling, not all particles may be reduced to the desired size. In such an event, the particles of the desired size may be separated and used in the practice of the present invention.

In another embodiment, a nanoparticulate composition may be formed by microprecipitation. This is a method of preparing stable dispersions of poorly soluble anidulafungin or an analogue thereof, in the presence of surface stabilizer(s) and one or more colloid stability-enhancing surface active agents free of any trace toxic solvents or solubilized heavy metal impurities. Such a method comprises, for example: (1) dissolving anidulafungin or an analogue thereof in a suitable solvent; (2) adding the formulation from step (1) to a solution comprising at least one surface stabilizer; and (3) precipitating the formulation from step (2) using an appropriate non-solvent. The method can be followed by removal of any formed salt, if present, by dialysis or diafiltration and concentration of the dispersion by conventional means.

In another embodiment of the invention, a nanoparticulate composition may be formed by homogenization. Exemplary homogenization methods are described in U.S. Pat. No. 5,510,118, for “Process of Preparing Therapeutic Compositions Containing Nanoparticles”, incorporated by reference herein. Such a method comprises dispersing particles comprising anidulafungin or an analogue thereof, in a liquid dispersion medium, followed by subjecting the dispersion to homogenization to reduce the particle size to the desired effective average particle size. The particles can be reduced in size in the presence of at least one surface stabilizer. The particles can be contacted with one or more surface stabilizers either before or after attrition. Other compounds, such as a diluent, can be added to the composition before, during, or after the size reduction process. Dispersions can be manufactured continuously or in a batch mode.

In another embodiment of the invention, a nanoparticulate composition may be formed by spray freezing into liquid (SFL). This technology comprises injecting an organic or organoaqueous solution of anidulafungin or an analogue thereof, and surface stabilizer(s) into a cryogenic liquid, such as liquid nitrogen. The droplets of the drug-containing solution freeze at a rate sufficient to minimize crystallization and particle growth, thus formulating nano-structured particles. Depending on the choice of solvent system and processing conditions, the particles can have varying particle morphology. In the isolation step, the nitrogen and solvent are removed under conditions that avoid agglomeration or ripening of the particles.

As a complementary technology to SFL, ultra rapid freezing (URF) may also be used to create equivalent nanostructured particles with greatly enhanced surface area.

URF comprises taking a water-miscible, anhydrous, organic, or organoaqueous solution of anidulafungin or an analogue thereof, and surface stabilizer(s) and applying it onto a cryogenic substrate. The solvent is then removed by means such as lyophilization or atmospheric freeze-drying with the resulting nanostructured particles remaining.

In another embodiment, a nanoparticulate composition may be made by template emulsion. Template emulsion creates nano-structured particles with controlled particle size distribution and rapid dissolution performance. The method comprises preparing an oil-in-water emulsion and then swelling it with a non-aqueous solution comprising anidulafungin or an analogue thereof and surface stabilizer(s). The size distribution of the particles is a direct result of the size of the emulsion droplets prior to loading of the emulsion with the drug. The particle size can be controlled and optimized in this process. Furthermore, through selected use of solvents and stabilizers, emulsion stability is achieved with no or suppressed Ostwald ripening. Subsequently, the solvent and water are removed, and the stabilized nano-structured particles are recovered. Various particle morphologies can be achieved by appropriate control of processing conditions.

In another embodiment, a nanoparticulate composition may be made by granulating in a fluidized bed an admixture comprising a nanoparticulate active agent dispersion, comprising at least one surface stabilizer, with a solution of at least one pharmaceutically acceptable water-soluble or water-dispersible excipient, to form a granulate.

According to an embodiment of the invention, solid or powder forms of nanoparticulate active agent dispersions can also be prepared by lyophilizing the liquid nanoparticulate active agent dispersion following particle size reduction.

In the lyophilization step, water is removed from the nanoparticulate active agent formulations after the dispersion is frozen and placed under vacuum, allowing the ice to change directly from solid to vapor without passing through a liquid phase. The lyophilization process consists of four interdependent processes: freezing, sublimation, the primary drying step, and desorption, which is the secondary drying step. Many lyophilizers can be used to achieve the lyophilization step of nanoparticulate active agent dispersions.

Suitable lyophilization conditions include, for example, those described in EP 0,363,365 (McNeil-PPC Inc.), U.S. Pat. No. 4,178,695 (A. Erbeia), and U.S. Pat. No. 5,384,124 (Farmalyoc), all of which are incorporated herein by reference. Typically, the nanoparticulate active agent dispersion is placed in a suitable vessel and frozen to a temperature of between about −5° C. to about −100° C. The frozen dispersion is then subjected to reduced pressure for a period of up to about 48 hours. The combination of parameters such as temperature, pressure, dispersion media, and batch size will impact the time required for the lyophilization process. Under conditions of reduced temperature and pressure, the frozen solvent is removed by sublimation yielding a solid, porous, immediate release solid dosage form having the nanoparticulate active agent distributed throughout.

Following sterilization, the lyophilized solid form can be formulated, for example, into a powder, tablet, suppository, or other solid dosage form, a powder can be formulated into an aerosol for nasal or pulmonary administration, or a powder can be reconstituted into a liquid dosage form, such as ocular drops, liquid nasal and pulmonary aerosols, ear drops, injectable compositions, etc.

One embodiment of the invention comprises a method for making a sterilized nanoparticulate anidulafungin composition comprising the steps of: mixing anidulafungin, at least one excipient, and at least one surface stabilizer in an aqueous medium containing milling media for a period of time and under conditions sufficient to provide a dispersion of particles of docetaxel having an effective average particle size of less than about 2000 nm and the at least one surface stabilizer adsorbed on the surface of the particles; removing the milling media from the dispersion; lyophilizing the dispersion to form a lyo; and sterilizing the lyo to produce a sterilized anidulafungin composition.

According to an embodiment of the invention, the solid nanoparticulate active agent particles are subjected to gamma radiation at ambient temperature, which remains relatively constant during the period of irradiation. Gamma radiation is applied in an amount sufficient to expose the pharmaceutical product to at least 25 kGray of irradiation. The total amount of gamma radiation that the solid nanoparticulate active agent is exposed to has been experimentally verified to: (1) render the active agent composition sterile, and (2) maintain the integrity of the nanoparticulate active agent composition. The application of the gamma radiation does not significantly degrade the active agent or reduce the active agent's efficacy. In this way, it is possible to provide products which meet cGMP requirements for sterile products without harming the active agent.

In a preferred aspect of the invention, the gamma radiation is applied in a preferred cumulative amount of about 5 kGray to about 50 kGray or less. Generally, the gamma radiation will normally be applied in a range of about 5 kGray to about 25 kGray or less.

One aspect of the invention is that upon reconstitution or redispersion after gamma irradiation, the terminally sterilized solid nanoparticulate active agent maintains its overall stability. Specifically the terminally sterilized solid nanoparticulate active agent maintains its redispersibility as evidenced by a retention of particle size, pH, osmolality, assay, and stabilizer concentration following redispersion of the solid in a liquid media.

### Methods of Treatment

In certain embodiments, the present invention also provides methods comprising the administration to a subject in need thereof an effective amount of a nanoparticulate composition comprising anidulafungin or an analogue thereof. As used herein, the term “subject” is used to mean an animal, preferably a mammal, including a human. The terms “patient” and “subject” may be used interchangeably. Thus, certain embodiments of the invention are directed to appropriate dosage forms useful in the administration of anidulafungin or an analogue thereof to a subject.

Certain aspects of the invention are directed to methods comprising the administration of an effective amount of a nanoparticulate composition comprising anidulafungin or an analogue thereof to a subject in need thereof. According to certain aspects of the invention, there are provided methods for the treatment of fungal infections or other parasitic infections.

Clinical trials have established the effectiveness of anidulafungin in treating (1) patients with candidemia and/or other forms of invasive candidiasis and (2) patients with esophageal candidiasis. In certain embodiments, nanoparticulate anidulafungin compositions of the invention may be administered to treat such patients.

In certain embodiments, the compositions of the invention may also be administered in conjunction with one or more additional active agents. These other active agents preferably include those useful for treatment of fungal or other parasitic infections as well as those agents useful for treating the adverse events that may be associated with anidulafungin treatment. Such active agents are preferably present in a manner, as determined by one skilled in the art, such that they do not interfere with therapeutic effect(s) of anidulafungin or an analogue thereof.

In human therapy, it is important to provide anidulafungin or analogue thereof dosage forms that deliver the required therapeutic amount of the drug in vivo, and that renders the drug bioavailable in a constant manner.

Bioavailability is the degree to which a drug becomes available to the target tissue after administration. Many factors can affect bioavailability, including the dosage form and various properties of the drug; for example, the dissolution rate. Poor bioavailability is a significant problem encountered in the development of pharmaceutical compositions, particularly those containing an active ingredient that is poorly soluble in water. Poorly water soluble drugs tend to be eliminated from the gastrointestinal tract before being absorbed into the circulation. Moreover, poorly water soluble drugs tend to be unsafe for intravenous administration techniques, which are used primarily in conjunction with fully soluble drug substances.

While the high therapeutic value of anidulafungin is recognized in the art, poorly soluble compounds such as anidulafungin are limited in their bioavailability upon oral administration and can be difficult to formulate as safe and effective products for other types of administration. Thus, there exists a need for formulations comprising anidulafungin which have improved oral bioavailability and thus improved efficacy and/or may be suitable for other types of administration, such as parenteral administration. An improvement in dissolution rate would enhance the bioavailability of anidulafungin, allowing a smaller dose to provide effective in vivo blood levels of the active agent. In addition, an enhanced dissolution rate could allow for a larger dose to be absorbed, which could increase the efficacy of the anidulafungin. An injectable nanoparticulate formulation of anidulafungin could eliminate the need for toxic co-solvents and enhance the efficacy of anidulafungin treatment. The present invention, which relates to nanoparticulate compositions comprising anidulafungin, addresses these concerns.

In addition to allowing for a smaller solid dosage form size, the nanoparticulate compositions of the present invention may exhibit increased bioavailability, and may require the administration of smaller doses of anidulafungin or analogue thereof, as compared to prior conventional, non-nanoparticulate compositions which comprise anidulafungin. In one embodiment of the invention, a nanoparticulate composition may have a bioavailability that is about 50% greater than anidulafungin or an analogue thereof, when administered in a conventional dosage form. In other embodiments, nanoparticulate compositions of the present invention may have a bioavailability that is about 40% greater, about 30% greater, about 20% greater, or about 10% greater than anidulafungin or an analogue thereof, when administered in a non-nanoparticulate dosage form.

The nanoparticulate composition may also have a desirable pharmacokinetic profile as measured following the initial dosage thereof to a mammalian subject. The desirable pharmacokinetic profile of the composition includes, but is not limited to: (1) a Cₘₐₓ for anidulafungin or an analogue thereof, when assayed in the plasma of a mammalian subject following administration that is preferably greater than the Cₘₐₓ for the same anidulafungin or an analogue thereof, when delivered at the same dosage by a non-nanoparticulate composition; and/or (2) an AUC for anidulafungin or an analogue thereof, when assayed in the plasma of a mammalian subject following administration that is preferably greater than the AUC for the same anidulafungin or an analogue thereof, when delivered at the same dosage by a non-nanoparticulate composition; and/or (3) a Tₘₐₓ for anidulafungin or an analogue thereof, when assayed in the plasma of a mammalian subject following administration that is preferably less than the Tₘₐₓ for the same anidulafungin or an analogue thereof, when delivered at the same dosage by a non-nanoparticulate composition.

In an embodiment of the present invention, a nanoparticulate composition may exhibit, for example, a Tₘₐₓ for anidulafungin or an analogue thereof contained therein which is not greater than about 90% of the Tₘₐₓ for the same anidulafungin or an analogue thereof, delivered at the same dosage by a non-nanoparticulate composition. In other embodiments of the present invention, the nanoparticulate composition of the present invention may exhibit, for example, a Tₘₐₓ for anidulafungin or an analogue thereof contained therein which is not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ for the same anidulafungin or an analogue thereof, delivered at the same dosage by a non-nanoparticulate composition.

In an embodiment of the present invention, a nanoparticulate composition of the present invention may exhibit, for example, a Cₘₐₓ for anidulafungin or an analogue thereof, contained therein which is at least about 50% of the Cₘₐₓ for the same anidulafungin or an analogue thereof, when delivered at the same dosage by a non-nanoparticulate composition. In other embodiments of the present invention, the nanoparticulate composition of the present invention may exhibit, for example, a Cₘₐₓ for anidulafungin or an analogue thereof contained therein which is at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ for the same anidulafungin or an analogue thereof, when delivered at the same dosage by a non-nanoparticulate composition.

In an embodiment of the present invention, a nanoparticulate composition of the present invention may exhibit, for example, an AUC for anidulafungin or an analogue thereof contained therein which is at least about 25% greater than the AUC for the same anidulafungin or an analogue thereof, when delivered at the same dosage by a non-nanoparticulate composition. In other embodiments of the present invention, the nanoparticulate composition of the present invention may exhibit, for example, an AUC for anidulafungin or an analogue thereof, contained therein which is at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC for the same anidulafungin or an analogue thereof, when delivered at the same dosage by a non-nanoparticulate composition.

### EXAMPLES

Examples have been set forth below for purposes of illustration and to describe the best mode of the invention at the present time. The scope of the invention is not to be in any way limited by the examples set forth herein.

### Example 1

This example describes the preparation of nanoparticles comprising anidulafungin.

Thirty grams of hydroxypropylcellulose (Klucel Type EF; Aqualon) is dissolved in 670 grams of deionized water using a continuous laboratory mixer. The hydroxypropylcellulose serves as a surface modifier. Three hundred grams of anidulafungin is then dispersed into the solution until a homogenous suspension is obtained. A laboratory scale media mill filled with polymeric grinding media is used in a continuous fashion until the mean particle size is approximately 200 nm as measured using a laser light scattering technique.

### Example 2

This example also describes the preparation of nanoparticles comprising anidulafungin.

Twenty five grams of polyvinylpyrrolidone (K29/32; BASF Corp.) is dissolved in 575 grams of deionized water using a continuous laboratory mixer. The polyvinylpyrrolidone serves as a surface modifier. Four hundred grams of anidulafungin is then dispersed into the solution until a homogenous suspension is obtained. A laboratory scale media mill filled with polymeric grinding media is used in a continuous fashion until the mean particle size is approximately 200 nm as measured using a laser light scattering technique.

It will be apparent to those skilled in the art that various modifications and variations can be made in the methods and compositions of the present inventions without departing from the spirit or scope of the invention. Thus, it is intended that the present invention cover the modification and variations of the inventions provided they come within the scope of the appended claims and their equivalents.

The terms and expressions which have been employed are used as terms of descriptions and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope on this invention.

In addition, where features or aspects of the invention are described in terms of Markush group or other grouping of alternatives, those skilled in the art will recognized that the invention is also thereby described in terms of any individual member or subgroup of members of the Markush group or other group.

Unless indicated to the contrary, all numerical ranges described herein include all combinations and subcombinations of ranges and specific integers encompassed therein. Such ranges are also within the scope of the described invention.

The disclosures of each patent, patent application and publication cited or described in this document are hereby incorporated herein by reference, in their entirety.

What is claimed is:

1. A composition comprising:
(a) particles comprising anidulafungin wherein the particles have an effective average particle size of less than about 2000 nm; and
(b) at least one surface stabilizer adsorbed on a surface of the particles.

2. The composition of claim 1, wherein said particles are in a form selected from the group consisting of crystalline, amorphous, semi-crystalline, semi-amorphous, and mixtures thereof.

3. The composition of claim 1, wherein the anidulafungin is selected from the group consisting of anidulafungin, salts of anidulafungin, derivatives of anidulafungin, conjugates of anidulafungin, hydrates of anidulafungin, polymorph of anidulafungin, analogues of anidulafungin, and mixtures thereof.

4. The composition of claim 1, wherein the effective average particle size is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, and less than about 50 nm.

5. The composition of claim 1, wherein the surface stabilizer is selected from the group consisting of a non-ionic surface stabilizer, an ionic surface stabilizer, a cationic surface stabilizer, a zwitterionic surface stabilizer, and an anionic surface stabilizer.

6. The composition of claim 1, wherein the at least one surface stabilizer is selected from the group consisting of povidone, cetyl pyridinium chloride, albumin, human serum albumin, bovine serum albumin, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, sodium deoxycholate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipids, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quaternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)4 ammonium chloride, lauryl dimethyl (ethenoxy)4 ammonium bromide, N-alkyl (C₁₂₋₁₈)dimethylbenzyl ammonium chloride, N-alkyl (C₁₄₋₁₈)dimethylbenzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUAT™, alkyl pyridinium salts; amines, amine salts, amine oxides, imide azolinium salts, protonated quaternary acrylamides, methylated quaternary polymers, and cationic guar.

7. The composition of claim 1, wherein:
(a) the at least one surface stabilizer is present in an amount selected from the group consisting of about 0.5% to about 99.999%, about 5.0% to about 99.9%, and about 10% to about 99.5%, by weight, based on the total combined dry weight of the anidulafungin and the at least one surface stabilizer, not including other excipients;
(b) the particles are present in an amount selected from the group consisting of about 99.5% to about 0.001%, about 95% to about 0.1%, and about 90% to about 0.5%, by weight, based on the total combined weight of the particles comprising the anidulafungin and the at least one surface stabilizer, not including other excipients; or
(c) the composition comprises a combination of (a) and (b).

8. The composition of claim 1 further comprising one or more pharmaceutically acceptable excipient, adjuvant, carrier, or a combination thereof.

9. The composition of claim 1, further comprising at least one excipient selected from the group consisting of a bulking agent, a crystal growth inhibitor, a free radical scavenger agent, and a redispersion agent.

10. The composition of claim 1, wherein the at least one excipient is present in the amount selected from the group consisting of from about 1 to about 50, about 1 to about 40, about 1 to about 30, about 1 to about 20, about 1 to about 15, about 1 to about 10, and about 1 to about 5, measured by % w/w.

11. The composition of claim 1, additionally comprising one or more active agents useful for the treatment of fungal infections.

12. The composition of claim 1, wherein the composition is formulated:
(a) for routes of administration selected from the group consisting of oral, pulmonary, rectal, opthalmic, colonic, parenteral, intracisternal, intravaginal, intraperitoneal, local, buccal, nasal, and topical administration;
(b) into a dosage form selected from the group consisting of liquid dispersions, solid dispersions, liquid-filled capsules, gels, aerosols, ointments, creams, lyophilized formulations, tablets, capsules, multi-particulate filled capsules, tablets composed of multi-particulates, compressed tablets, and capsules filled with enteric-coated beads of the active agent;
(c) into a dosage form selected from the group consisting of controlled release formulations, fast melt formulations, delayed release formulations, extended release formulations, pulsatile release formulations, and mixed immediate release and controlled release formulations; or
(d) into any combination of (a), (b), and (c).

13. The composition of claim 12, wherein the composition is an oral formulation.

14. The composition of claim 12, wherein the composition is an injectable formulation.

15. The composition of claim 12, wherein the composition is formulated as an injectable subcutaneous or intramuscular depot for long term release.

16. The composition of claim 15, wherein release occurs over a period from about 1 week to about 4 weeks.

17. The composition of claim 12, wherein the composition is formulated for ocular administration.

18. The composition of claim 12, wherein the composition is formulated for pulmonary administration.

19. The composition of claim 1, wherein the composition has greater bioavailability as compared to conventional compositions comprising anidulafungin.

20. The composition of claim 1 wherein: (a) the Tₘₐₓ of the composition, when assayed in the plasma of a mammalian subject following administration, is less than the Tₘₐₓ for a non-nanoparticulate composition comprising the same anidulafungin, administered at the same dosage; (b) the Cₘₐₓ of the composition, when assayed in the plasma of a mammalian subject following administration, is greater than the Cₘₐₓ for a non-nanoparticulate composition comprising the same anidulafungin, administered at the same dosage; (c) the AUC of the composition, when assayed in the plasma of a mammalian subject following administration, is greater than the AUC for a non-nanoparticulate composition comprising the same anidulafungin, administered at the same dosage; or (d) any combination of (a), (b) and (c).

21. A method for making a nanoparticulate composition comprising the step of contacting at least one active agent selected from the group consisting of anidulafungin, salts of anidulafungin, derivatives of anidulafungin, conjugates of anidulafungin, hydrates of anidulafungin, polymorphs of anidulafungin, and analogues of anidulafungin, with at least one surface stabilizer for a period of time and under conditions sufficient to provide a nanoparticulate composition having an effective average particle size of less than about 2000 nm.

22. The method of claim 20, wherein the composition comprises particles having an effective average particle size selected from the group consisting of less than about 2000 nm, less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1 micron, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, and less than about 50 nm.

23. A method of treating a subject in need of anidulafungin comprising administering to the subject an effective amount of a composition comprising:
(a) particles comprising anidulafungin, a salt, derivative, conjugate, hydrate, polymorph or analogue thereof, wherein the particles have an effective average particle size of less than about 2000 nm; and
(b) at least one surface stabilizer adsorbed on a surface of the particles.

24. The method of claim 22, wherein the composition is administered by injection.

25. The method of claim 22, wherein the composition is administered as an injectable subcutaneous or intramuscular depot for long term release.

26. The method of claim 24, wherein release occurs over a period from about 1 week to about 4 weeks.

27. The method of claim 22, wherein the composition is administered by ocular administration.

28. The method of claim 22, wherein the composition is administered by pulmonary administration.

29. A method of treating a fungal infection in a patient comprising the step of administering to the patient an effective amount of the composition of claim 1.
